(19) United States
(12) Patent Application Publication
(10) Pub. No.: US 2024/0047006 A1
(43) Pub. Date: Feb. 8, 2024

(54) ARTIFICIAL INTELLIGENCE SYSTEM AND METHOD FOR DESIGNING PROTEIN SEQUENCES

(71) Applicant: Lurong Pan, Vestavia Hill, AL (US)

(72) Inventor: Lurong Pan, Vestavia Hill, AL (US)

(21) Appl. No.: 18/481,286

(22) Filed: Oct. 5, 2023

Related U.S. Application Data
(63) Continuation-in-part of application No. 17/811,091, filed on Jul. 7, 2022.

(51) Int. Cl. G16B 15/30

(52) U.S. Cl. CPC G16B 15/30

(57) ABSTRACT

A system and method for collaborative smart evidence gathering and investigation for incident response attack surface management and forensics in a computing environment is disclosed. The system obtains evidence data from multiple sources with various entry points, capturing contextual information. Further, the system processes the data using an artificial intelligence (AI) root cause analysis, graph augmented retrieval, semantic classifier, meaning extraction, and causal discovery model. Furthermore, the system performs similarity analysis to assess evidence quality, sufficiency, and completeness. Based on the evaluation, the system determines appropriate actions to be taken on the processed evidence data. Additionally, the system executes the actions to resolve the incidents effectively by using a smart expert system, a human agent participation, or an AI co-pilot, as a first-class investigator and collaborator in the process.

### CROSS-REFERENCE TO RELATED APPLICATIONS

The present application is a continuation-in-part of U.S. patent application Ser. No. 17/811,091, filed on Jul. 7, 2022, and titled “A SYSTEM AND METHOD OF ANTIBODY/MACROMOLECULE DRUG AFFINITY MODIFICATION”, which claims priority from Chinese Patent Application 2022105370156 filed on May 17, 2022, and titled “A SYSTEM AND METHOD OF ANTIBODY/MACROMOLECULE DRUG AFFINITY MODIFICATION”; each of the above-identified applications is fully incorporated herein by reference.

### TECHNICAL FIELD

Embodiments of the present disclosure generally relate to artificial intelligence (AI) based systems and more particularly to an artificial intelligence (AI) system and a method for designing protein sequences.

### BACKGROUND

Generally, proteins are vital for biological functions, and designing or modifying the proteins is crucial for pharmaceuticals and biotechnology. Computational protein language models, especially generative models, have emerged as a promising solution. The language models learn from vast datasets of natural protein sequences and may generate new designs or evaluate sequence variants for fitness, offering an effective and efficient approach to protein engineering. Currently, there has been a profound exploration of artificial intelligence (AI) and machine learning to master the complexities of language and the design of functional proteins. Language, as a highly intricate system of human expression governed by grammatical rules, has long posed a significant challenge for AI algorithms to comprehend and manipulate effectively. Simultaneously, in the field of molecular biology and bioengineering, there has been a growing interest in designing proteins with specific functions for various applications.

Conventionally, methods provide pre-trained language models (PLMs) based on transformer architectures, to address natural language processing (NLP) tasks. Furthermore, the scaling of these models to larger parameters enables in-context learning, setting the stage for large language models (LLMs). Further, another conventional method provides generative protein language models in designing novel proteins with desired functions. However, existing models face challenges in generating proteins from specific families of interest or necessitate extensive training on family-specific data, limiting their adaptability across different protein families. Another conventional method provides a protein evolutionary transformer (PoET), which is a generative model for designing new proteins with specific functions. The PoET learns to generate sets of related proteins across diverse protein families. However, the conventional methods may not specifically address the complexities of protein sequence design and may not incorporate a deep understanding of biological contexts, such as protein-protein interactions or immunogenicity, which are crucial in pharmaceutical and biotechnological applications.

Consequently, there is a need for an improved an artificial intelligence system and a method for designing protein sequences to address at least the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

An aspect of the present disclosure provides an artificial intelligence (AI) system for designing protein sequences. The AI system trains a generative artificial intelligence (AI) model with pre-stored biologics assay results using a task specific dataset and a large language model. The task specific dataset comprises a plurality of protein sequences. Further, the AI system trains a reward model based on in vitro and in silico evidence. Further, the AI system generates target specific protein sequences based on the trained generative AI model. Additionally, the system calculates a reward score for each of the generated target specific protein sequences based on the trained reward model and a reinforcement learning model. Further, the AI system generates a ranked list of the generated target specific protein sequences based on the calculated reward score. Furthermore, output the generated ranked list of the target specific protein sequences on a user device.

Another aspect of the present disclosure provides an artificial intelligence (AI) method for designing protein sequences. Furthermore, the AI method includes training a generative artificial intelligence (AI) model with pre-stored biologics assay results using a task specific dataset and a large language model. The task specific dataset comprises a plurality of protein sequences. Further, the AI method includes training a reward model based on in vitro and in silico evidence. Furthermore, the AI method includes generating a target specific protein sequence based on the trained generative AI model. Additionally, the AI method includes calculating a reward score for each of the generated target specific protein sequences based on the trained reward model and a reinforcement learning model. Further, the AI method includes generating a ranked list of the generated target specific protein sequences based on the calculated reward score. Furthermore, the AI method includes outputting the generated ranked list of the target specific protein sequences on a user device.

Yet another aspect of the present disclosure provides a non-transitory computer-readable storage medium having instructions stored therein. When executed by one or more hardware processors, cause the one or more hardware processors to train a generative artificial intelligence (AI) model with pre-stored biologics assay results using a task specific dataset and a large language model. The task specific dataset comprises a plurality of protein sequences. The one or more hardware processors train a reward model based on in vitro and in silico evidence. Further, the one or more hardware processors generate a target specific protein sequence based on the trained generative AI model. Additionally, the one or more hardware processors calculate a reward score for each of the generated target specific protein sequences based on the trained reward model and a reinforcement learning model. Further, the one or more hardware processors generate a ranked list of the generated target specific protein sequences based on the calculated reward score. Furthermore, the one or more hardware processors output the generated ranked list of the target specific protein sequences on a user device.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 illustrates an exemplary block diagram representation of a network architecture implementing an artificial intelligence system for designing protein sequences, in accordance with an embodiment of the present disclosure:

FIG. 2 illustrates an exemplary block diagram representation of a computer implemented system, such as those shown in FIG. 1, capable of designing protein sequences, in accordance with an embodiment of the present disclosure:

FIG. 3A illustrates an exemplary flow diagram representation of a protein sequence generation using large language models (LLMs), in accordance with an embodiment of the present disclosure;

FIG. 3B illustrates an exemplary flow diagram representation of a protein molecule affinity maturation using protein language models (PLMs), in accordance with an embodiment of the present disclosure;

FIG. 4A illustrates an exemplary flow diagram representation of method for modifying virtual antibody/protein affinity, in accordance with an embodiment of the present disclosure;

FIG. 4B illustrates an exemplary flow diagram representation of method for modifying virtual antibody/protein affinity modification in a scenario, in accordance with an embodiment of the present disclosure;

FIG. 5 illustrates a flow chart depicting a method of designing protein sequences, in accordance with the embodiment of the present disclosure; and

FIG. 6 illustrates an exemplary block diagram representation of a hardware platform for implementation of the disclosed system, according to an example embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module includes dedicated circuitry or logic that is permanently configured (within a special-purpose processor) to perform certain operations. In another embodiment, a “module” or s “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

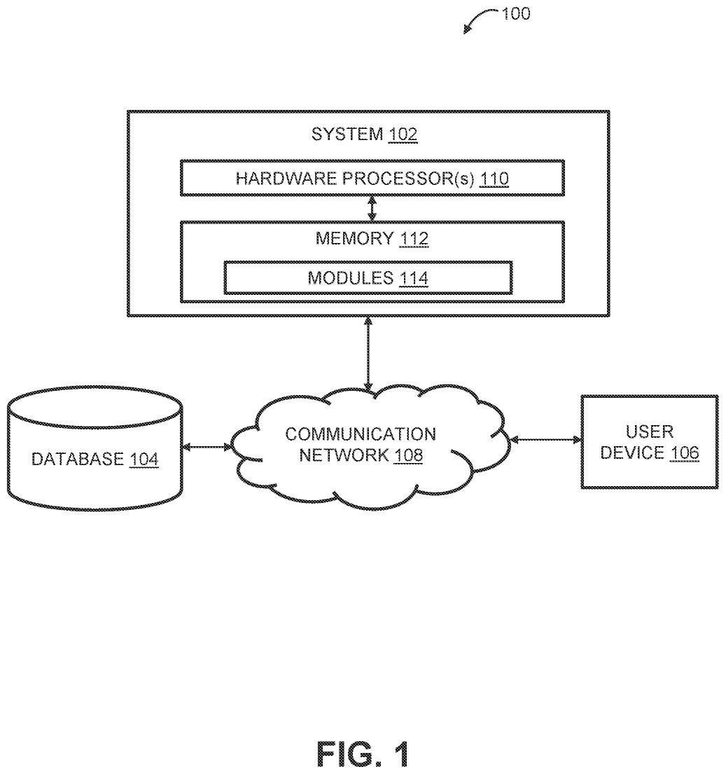

Referring now to the drawings, and more particularly to FIG. 1 through FIG. 6, where similar reference characters denote corresponding features consistently throughout the figures, there are shown preferred embodiments and these embodiments are described in the context of the following exemplary system and/or method.

FIG. 1 illustrates an exemplary block diagram representation of a network architecture 100 implementing an artificial intelligence system 102 for designing protein sequences, in accordance with an embodiment of the present disclosure. According to FIG. 1, the network architecture 100 includes a system 102, a database 104, and one or more user devices 106. The one or more user devices 106 may be associated with one or more users, and communicatively coupled to the system 102 via a communication network 108. In an exemplary embodiment of the present disclosure, the user devices 106 may include a laptop computer, desktop computer, tablet computer, smartphone, wearable device, a digital camera, and the like. Further, the communication network 108 may be a wired network or a wireless network. The system 102 may be at least one of, but not limited to, a central server, a cloud server, a remote server, an electronic device, a portable device, and the like. Further, the system 102 may be communicatively coupled to the database 104, via the communication network 108. The database 104 may include, but is not limited to, task specific dataset, template sequence information, biologics, biologics assay results, protein sequences data, rewards data, ranked list of the generated target specific protein sequences, reward score, affinity, immunogenicity, stability, toxicity, enzymatic activity for therapeutic or non-therapeutic use, protein-protein interaction affinity, protein stability, immunogenicity, toxicity results, plurality of tokens, label ranked results, vitro evidence, in silico evidence, any other data, and combinations thereof. The database 104 may be any kind of databases/repositories such as, but are not limited to, relational repositories, dedicated repositories, dynamic repositories, monetized repositories, scalable repositories, cloud repositories, distributed repositories, any other repositories, and combination thereof.

Further, the user device 106 may be associated with, but not limited to, a user, an individual, an administrator, a vendor, a technician, a worker, a specialist, a healthcare worker, an instructor, a supervisor, a team, an entity, an organization, a company, a facility, a bot, any other user, and combination thereof. The entities, the organization, and the facility may include, but are not limited to, a hospital, a healthcare facility, an exercise facility, a laboratory facility, an e-commerce company, a merchant organization, an airline company, a hotel booking company, a company, an outlet, a manufacturing unit, an enterprise, an organization, an educational institution, a secured facility, a warehouse facility, a supply chain facility, any other facility and the like. The user device 106 may be used to provide input and/or receive output to/from the system 102, and/or to the database 104, respectively. The user device 106 may present to the user one or more user interfaces for the user to interact with the system 102 and/or to the database 104 for protein sequences designing need. The user device 106 may be at least one of, an electrical, an electronic, an electromechanical, and a computing device. The user device 106 may include, but is not limited to, a mobile device, a smartphone, a personal digital assistant (PDA), a tablet computer, a phablet computer, a wearable computing device, a virtual reality/augmented reality (VR/AR) device, a laptop, a desktop, a server, and the like.

Further, the system 102 may be implemented by way of a single device or a combination of multiple devices that may be operatively connected or networked together. The system 102 may be implemented in hardware or a suitable combination of hardware and software. The system 102 includes one or more hardware processor(s) 110, and a memory 112. The memory 112 may include a plurality of modules 114. The system 102 may be a hardware device including the hardware processor 110 executing machine-readable program instructions for designing protein sequences. Execution of the machine-readable program instructions by the hardware processor 110 may enable the proposed system 102 to designing protein sequences. The “hardware” may comprise a combination of discrete components, an integrated circuit, an application-specific integrated circuit, a field-programmable gate array, a digital signal processor, or other suitable hardware. The “software” may comprise one or more objects, agents, threads, lines of code, subroutines, separate software applications, two or more lines of code, or other suitable software structures operating in one or more software applications or on one or more processors.

The one or more hardware processors 110 may include, for example, microprocessors, microcomputers, microcontrollers, digital signal processors, central processing units, state machines, logic circuits, and/or any devices that manipulate data or signals based on operational instructions. Among other capabilities, hardware processor 110 may fetch and execute computer-readable instructions in the memory 112 operationally coupled with the system 102 for performing tasks such as data processing, input/output processing, and/or any other functions. Any reference to a task in the present disclosure may refer to an operation being or that may be performed on data.

Though few components and subsystems are disclosed in FIG. 1, there may be additional components and subsystems which is not shown, such as, but not limited to, ports, routers, repeaters, firewall devices, network devices, databases, network attached storage devices, servers, assets, machinery, instruments, facility equipment, emergency management devices, image capturing devices, sensors, any other devices, and combination thereof. The person skilled in the art should not be limiting the components/subsystems shown in FIG. 1. Although FIG. 1 illustrates the system 102, and the user device 106 connected to the database 104, one skilled in the art can envision that the system 102, and the user device 106 can be connected to several user devices located at various locations and several databases via the communication network 108.

Those of ordinary skilled in the art will appreciate that the hardware depicted in FIG. 1 may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the system 102 as is unique to the present disclosure or necessary for an understanding of the present disclosure is depicted and described. The remainder of the construction and operation of the system 102 may conform to any of the various current implementations and practices that were known in the art.

In an exemplary embodiment, the system 102 may train a generative artificial intelligence (AI) model with pre-stored biologics assay results using a task specific dataset and a large language model. The task specific dataset comprises a plurality of protein sequences. The biological assay results comprise protein-protein interaction affinity, protein stability, immunogenicity, and toxicity results. Further, the system 102 may train a reward model based on in vitro and in silico evidence. Furthermore, the system 102 may generate target specific protein sequences based on the trained generative AI model. Additionally, the system 102 may calculate a reward score for each of the generated target specific protein sequences based on the trained reward model and a reinforcement learning model. Further, the system 102 may generate a ranked list of the generated target specific protein sequences based on the calculated reward score. The ranked list of the target specific protein sequences is generated based on the predicted properties and suitability for specific applications in biological research and drug discovery. Furthermore, the system 102 may output the generated ranked list of the target specific protein sequences on a user device.

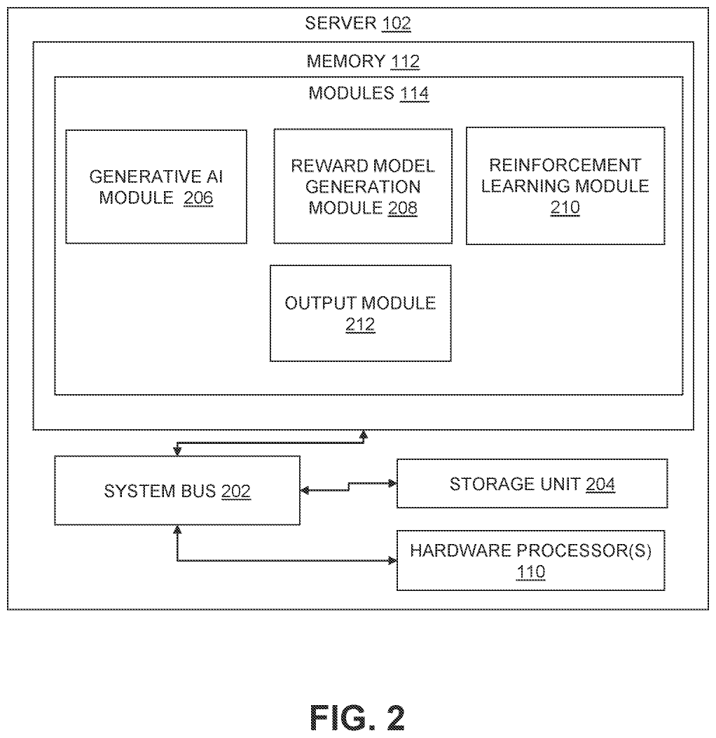

FIG. 2 illustrates an exemplary block diagram representation of a computer implemented system 102, such as those shown in FIG. 1, capable of designing protein sequences, in accordance with an embodiment of the present disclosure. The system 102 may also function as a computer-implemented system/server (hereinafter referred to as the system 102). The system 102 comprises the one or more hardware processors 110, the memory 112, and a storage unit 204. The one or more hardware processors 110, the memory 112, and the storage unit 204 are communicatively coupled through a system bus 202 or any similar mechanism. The memory 112 comprises a plurality of modules 114 in the form of programmable instructions executable by the one or more hardware processors 110.

Further, the plurality of modules 114 includes a generative artificial intelligence (AI) module 206, a reward model generation module 208, a reinforcement learning module 210, and an output module 212.

The one or more hardware processors 110, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 110 may also include embedded controllers, such as generic or programmable logic devices or arrays, application-specific integrated circuits, single-chip computers, and the like.

The memory 112 may be a non-transitory volatile memory and a non-volatile memory. The memory 112 may be coupled to communicate with the one or more hardware processors 110, such as being a computer-readable storage medium. The one or more hardware processors 110 may execute machine-readable instructions and/or source code stored in the memory 112. A variety of machine-readable instructions may be stored in and accessed from the memory 112. The memory 112 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory 112 includes the plurality of modules 114 stored in the form of machine-readable instructions on any of the above-mentioned storage media and may be in communication with and executed by the one or more hardware processors 110.

The storage unit 204 may be a cloud storage or a device information repository such as those shown in FIG. 1. The storage unit 204 may store, but is not limited to, task specific dataset, template sequence information, biologics, biologics assay results, protein sequences data, rewards data, ranked list of the generated target specific protein sequences, reward score, affinity, immunogenicity, stability, toxicity, enzymatic activity for therapeutic or non-therapeutic use, protein-protein interaction affinity, protein stability, immunogenicity, toxicity results, plurality of tokens, label ranked results, vitro evidence, in silico evidence, any other data, any other data, and combinations thereof. The storage unit 204 may be any kind of databases/repositories such as, but are not limited to, relational repositories, dedicated repositories, dynamic repositories, monetized repositories, scalable repositories, cloud repositories, distributed repositories, any other repositories, and combination thereof.

In an exemplary embodiment, the generative artificial intelligence (AI) module 206 may train a generative artificial intelligence (AI) model with pre-stored biologics assay results using a task specific dataset and a large language model. The task specific dataset comprises a plurality of protein sequences. The biological assay results comprise protein-protein interaction affinity, protein stability, immunogenicity, and toxicity results.

In an embodiment, for training the generative artificial intelligence (AI) model with pre-stored biologics assay results using the task specific dataset and a large language model, the generative AI module 206 may train a large language model with a plurality of protein sequences comprising the task specific dataset. The plurality of protein sequences is assigned with a plurality of tokens. Further, the generative AI module 206 may re-train the trained large language model with a pre-stored biological assay results using a supervised learning model. The pre-stored biological assay results comprise protein-protein interaction affinity, protein stability, immunogenicity, and toxicity results. Further, the generative AI module 206 may train the generative artificial intelligence (AI) model with pre-stored biologics assay results using the task specific dataset and the re-trained large language model.

In an embodiment, the reward model generation module 208 may train a reward model based on in vitro and in silico evidence.

In an embodiment, to train the reward model based on in vitro and in silico evidence, the reward model generation module 208 may sample a plurality of historical input and output datasets. Further, the reward model generation module 208 may generate a label ranked results for reward model by performing wet lab analysis on the sampled plurality of historical input and output datasets. Furthermore, the reward model generation module 208 may generate the reward model based on in vitro evidence, in silico evidence and the label ranked results.

In an embodiment, the reinforcement learning module 210 may generate target specific protein sequences based on the trained generative AI model. Additionally, the reinforcement learning module 210 may calculate a reward score for each of the generated target specific protein sequences based on the trained reward model and a reinforcement learning model. Further, the reinforcement learning module 210 may generate a ranked list of the generated target specific protein sequences based on the calculated reward score. The ranked list of the target specific protein sequences is generated based on the predicted properties and suitability for specific applications in biological research and drug discovery. Furthermore, the reinforcement learning module 210 may output the generated ranked list of the target specific protein sequences on a user device.

In an embodiment, for generating target specific protein sequences based on the trained generative AI model, the reinforcement learning module 210 may input template sequence information of antibody/macromolecular drugs, modification requirements of single/multi-targets of antibody/macromolecular drugs and optional user-defined screening requirements to generate target specific protein sequences. Further, the reinforcement learning module 210 may perform corresponding partial or exhaustive numeration of sequence in a part of the full variable range to obtain a mutation library and perform sequence-based affinity prediction on the mutation library based on the trained generative AI model, to obtain the specific protein sequences of the modified antibody/macromolecular drug. Additionally, the reinforcement learning module 210 may generate the target specific protein sequences of the candidate antibody/macromolecular drug according to the target specific protein sequences of the modified antibody/macromolecular drug.

In an embodiment, the system 102 may generate, but not limited to, protein functional predictions comprising affinity, immunogenicity, stability, toxicity, enzymatic activity for therapeutic or non-therapeutic use, and the like. In an embodiment, the system 102 may optimize the supervised learning model based on target-specific biological assay results. In an embodiment, the system 102 may self-update the reinforcement learning model, the supervised learning model and the generative AI model based on the generated target specific protein sequences and the generated ranked list of the target specific protein sequences.

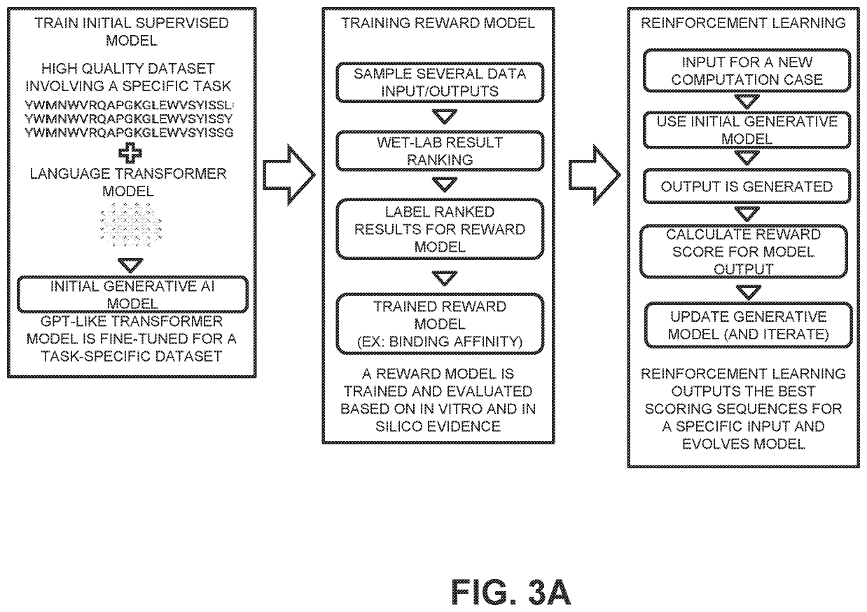

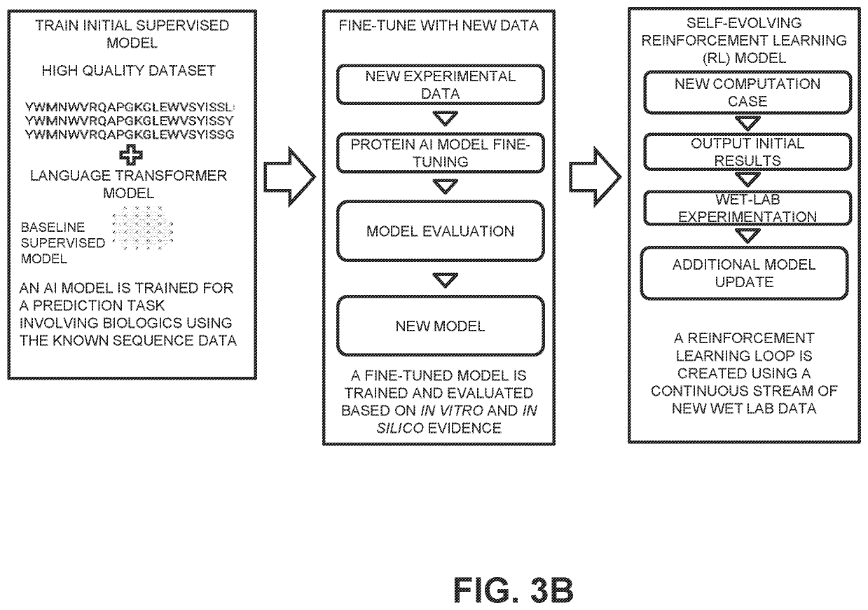

FIG. 3A illustrates an exemplary flow diagram representation of a protein sequence generation using large language models (LLMs), in accordance with an embodiment of the present disclosure. Consider a scenario of 20 amino acids combined to form proteins. The natural language sentences may be used for model proteins using generative pre-trained transformer (GPT) methodologies. Further, the system 102 may train the initial supervised model. High quality dataset involving a specific task may be used for the training. Further, using a language transformer model, an initial generative AI model may be generated. The GPT transformer model may be fine-tuned for a task-specific dataset. Further, the system 102 may train reward model. To train, the system 102 may retrieve sample several data input/outputs. Further, the system 102 may rank wet-lab results, label ranked results for reward model. GPT training methodology using wet-lab results for a ranked reward function.

The system 102 may output trained reward model (e.g., binding affinity) based on the sample data inputs and label ranked results. A reward model may be trained and evaluated based on in vitro and in silico evidence. Further, the system 102 may perform reinforcement learning (RL) of the model. To perform, RL, the system 102 may input for a new computation case, to the model. Further, the system 102 may use the initial generative model, and generate output. Based on the output, the system 102 may calculate reward score for model output and update generative model (and iterate). Reinforcement learning outputs the best scoring sequences for a specific input and evolves model.

FIG. 3B illustrates an exemplary flow diagram representation of a protein molecule affinity maturation using protein language models (PLMs), in accordance with an embodiment of the present disclosure. Consider a scenario of 20 amino acids combined to form proteins. Further, natural language sentences are used to model proteins using natural language processing (NLP) methodologies. The system 102 may train initial supervised model with high quality dataset to generate language transformer model. The language transformer model may be based on baseline supervised model. An AI model may be trained for a prediction task involving biologics using known sequence data.

Further, the system 102 may fine-tune with new data. The new experimental data may be obtained for generating a fine-tune protein AI model. Further, the system 102 may perform model evaluation and then generate a new model. A fine-tuned model may be trained and evaluated based on in vitro and in silico evidence. Further, the system 102 may enable self-evolving of RL model. The system 102 may input a new computation case for the RL model. Further the system 102 may output initial results using the RL model. The initial results may be used to perform wet-lab experimentation, and then additional model update. The reinforcement learning loop is created using a continuous stream of new wet lab data. Then the model may be a final evolving model.

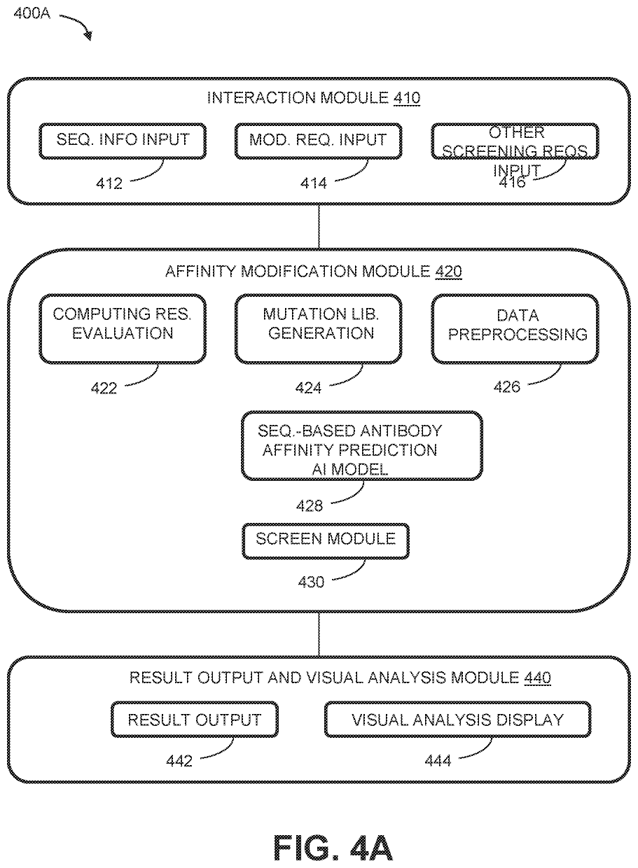

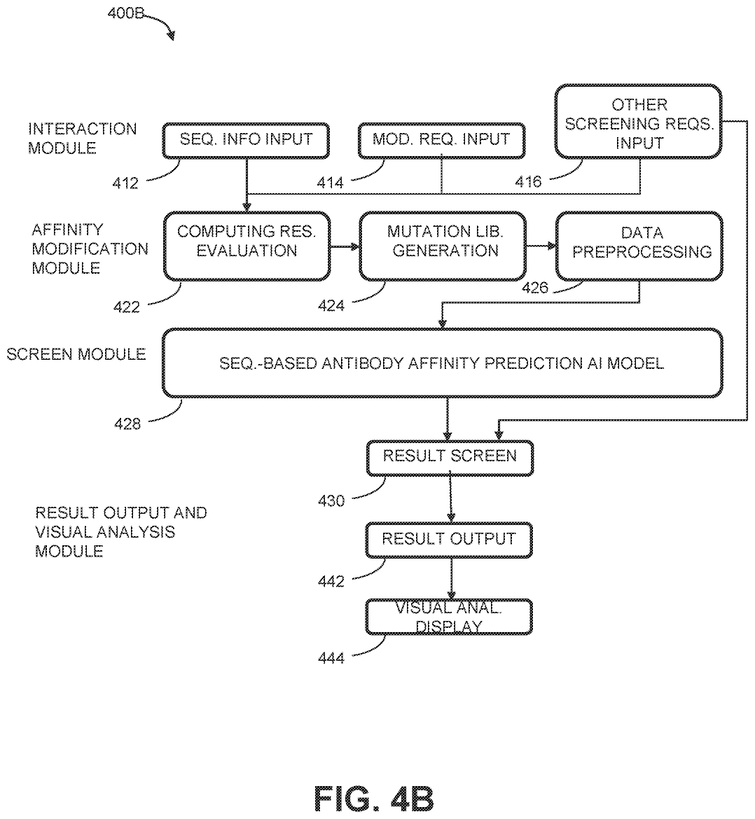

FIG. 4A illustrates an exemplary flow diagram representation of method 400A for modifying virtual antibody/protein affinity, in accordance with an embodiment of the present disclosure. In the current single/multi-target affinity modification module 120 of antibody drugs or macromolecular drugs, the following are several common scenarios that lead to new problems, and the corresponding solutions adopted to solve the new problems. In the aspects of computational-assisted affinity engineering, the traditional computer simulation methods calculate the antibody-antigen binding strengths according to the atomic chemical properties such as polarity and charge. With the rapid development of machine learning and deep learning research fields, AI-based algorithms and techniques have been explored for application in antibody development. Then, the prediction of interacting contacts was then investigated, and binding predictions were made by using surface-based geometric features; and then a topology-based network tree may be employed to predict binding affinity changes based on the 3D structure of the complex. A long-term and short-term memory model 128 for antigen-specific affinity prediction was then trained on an in-computer sequence library. Thereafter, the mutations in CDR-H3 were used for sequence-based deep learning antibody design for computer antibody affinity maturation. To sum up, the above computational-based antibody affinity prediction methods either rely on the 3D structural information of antibodies and antigens, or rely on artificially defined chemical characteristics, or rely on the determined epitope information, which limits the application of the above tools to unknown structural targets. In addition, the verification manners of the affinity model of calculation and screen, using a screen module 430, above are mainly divided into two categories, one is to train and test the backtracking data 426 of a single target, and the other is to include the known target data 426 in the training data 426 in the test data 426. The above two methods are difficult to directly reflect the generalization ability of the model in other antigen-antibody affinities, which limits the practical application in pharmaceutical process.

In addition, both traditional experimental and computational assisted cannot avoid the limited space for antibody modification, the modification methods partially or completely depend on the antigen/target structural information. The experimental construction or model construction aimed at one or a certain type of target, and the time cost high, the cost of downstream experiments is high, and the design methods are not universal, and the like.

In view of the above problems, the purpose of the present invention is to overcome the following shortcomings: the traditional antibody affinity maturation technology adopts random mutation or computer-assisted site-directed mutation (such as point mutation only for CDR-H3 region of antibody) to generate antibody mutation library 424, which has high experimental construction cost and long experimental period. At the same time, limited by the experimental cost and calculation methods, the above methods have limited imagination space and high randomness for molecular modification, and it is difficult to directly confirm the improvement degree of affinity through screening, using the screen module 430, so that the cost of verifying affinity in downstream experiments is higher.

In view of the above shortcomings, the invention aims to solve the limitations of traditional artificial design methods and traditional computer-aided methods, screen the amino acid sequence of antibody/fusion protein up to one billion-level mutation spaces, significantly improve the screening hit rate of high affinity antibodies/macromolecules, and greatly reduce the time and screening cost of downstream experiments. In addition, the invention does not depend on the structural information or epitope information of antigen/target and can directly optimize the virtual affinity maturation of antibody/macromolecule from the amino acid sequence level, which plays an important auxiliary role in macromolecular drug design of new target. More importantly, the virtual affinity module of the invention adopts a fully automatic calculation process, has a fast-screening speed (the screening of one billion-level mutation spaces takes hours as a unit), and can simultaneously screen for multiple affinity modification conditions of multiple targets.

FIG. 4B illustrates an exemplary flow diagram representation of method for modifying virtual antibody/protein affinity modification in a scenario, in accordance with an embodiment of the present disclosure. The first aspect of the present invention provides an antibody/macromolecular drug single/multi-target affinity modification system. The affinity modification system comprises, an interaction module 410, the interaction module 410 is set to: input template sequence information 412 of antibody/macromolecular drugs, modification requirements of single/multi-targets of antibody/macromolecular drugs and optional user-defined screening requirements 416 to generate interaction antibody/macromolecular drug sequence information 412.

The affinity modification module 420, the affinity modification module 420 is set to: according to the interaction antibody/macromolecular drug sequence information 412, perform partial or exhaustive numeration of possible sequence in a part of the full variable range to obtain a mutation library 424, and perform sequence-based affinity prediction on the mutation library 424 based on a deep learning model, so as to obtain the sequence information 412 of the modified antibody/macromolecular drug.

An output module 440, the output module 440 is designed to: according to the sequence information 412 of the modified antibody/macromolecular drug, output the sequence information 412 of the candidate antibody/macromolecular drug. In a preferred embodiment of the present invention, in the affinity design module, the single quantity level of the mutation library 424 is not less than 1010. In a preferred embodiment of the present invention, in the affinity design module, the variable range includes one or more variable regions, variable spaces, variable number of sites or combinations thereof.

In an embodiment of the present invention, in the interaction module 410, the template sequence information 412 of the antibody/macromolecular drug includes antigen/antibody template sequence, protein/protein template sequence, or protein/polypeptide template sequence of the antibody/macromolecular drug. In an embodiment of the present invention, in the interaction module 410, in the modification requirements of single/multiple targets of the antibody/macromolecular drug,

Marking or specifying the variable range; and/or defining the modification direction. In an embodiment of the present invention. The output module 440 further comprises a visual analysis display module 444. In a preferred embodiment of the present invention, the visual analysis display module 444 provides the complete sequence information 412 of the sequence information 412 of the candidate antibody/macromolecular drug. In a preferred embodiment of the present invention, the visual analysis display module 444 further comprises a comparative analysis of the template sequence information 412 of the antibody/macromolecular drug and the sequence information 412 of the candidate antibody/macromolecular drug in a variable range.

In an embodiment of the present invention, an automatic virtual antibody/macromolecule affinity maturation technology based on data 426 driven and artificial intelligence algorithm is provided.

The invention includes: an affinity maturation interaction module 410, an affinity maturation design module based on artificial intelligence, and an affinity maturation visual analysis display module 444. The interaction module 410 requires the user to input antigen/antibody template sequence (or protein/protein, protein/polypeptide), wherein, the antigen/target can be multiple sequences. This module allows users to mark and specify the variable region (variable region) and the variable space range of interest and define the modification direction of a single target one by one (affinity enhancement or weakening). It also allows to define the number of antibody sequences produced by virtual screening according to the user's situation (such as the estimated cost of the downstream experiment).

Sequence information 412 (and other user-defined information) is input from the interaction module 410 to the calculation module, according to the upstream information, the affinity maturation design module exhausts the variable space range of antibodies to generate antibody mutation library 424. The single mutation library 424 level can reach 1010. The calculation module preprocesses the sequence information 412 in the library one by one and calculates and records the affinity of antibody antigen based on the deep learning model. Finally, the qualified antibody sequences are screened and output according to the user-defined screening conditions.

All antibody/protein candidate modified sequences generated by the design module enter the visual analysis display module 444. The visualization module provides mutation sites comparison of template sequences and candidate modification sequences, statistical charts of mutation sites, and the display of mutation sites thermal map, and the like.

The affinity modification module 420 based on artificial intelligence of the present invention includes an affinity modification interaction module 410, an affinity modification design module based on artificial intelligence, and a result output 442 and visual analysis display module 444. The target users of the invention are the biological drugs/antibody drugs researchers.

The design/operation steps of affinity modification module 420 are as follows. S1. The interaction module 410 is the user input interface, allowing the user to input antigen sequence, antibody sequence (or target protein/drug protein sequence). Among them, the antigen/target can be multiple sequences, and the modification direction of a single target can be defined one by one (affinity enhancement or weakening). This module allows users to mark and specify the variable region (variable region) and variable space range of interest, define the modification direction (affinity enhancement or weakening), and define the number of antibody sequences produced by virtual screening according to the user's situation (such as the estimated cost of the downstream experiment). For example, to optimize the antibody template of a certain antigen, it is necessary to fill in the complete sequence information 412 of the antibody antigen, and fill in the modification requirements of antibody affinity, that is, to enhancement or weakening. At the same time, users can choose to limit the mutation site to a certain position range, such as the CDR-H3 region of the antibody. The input module allows users to customize multiple regions of interest. At the same time, users can also define the number of mutation sites, and can choose single-point mutation, double-point mutation or multi-point mutation (3-5 points). Finally, the user can define the number of candidate antibody sequences given by the module according to the actual situation (such as the estimated cost of the downstream experiment).

S2, the calculation module receives the amino acid sequence information 412, the modification direction information and other user-defined information provided by the interaction module 410. According to the upstream information, the affinity maturation design module evaluates the mutation space of the antibody. If the mutation space exceeds the calculated maximum upper limit of 1010, it will prompt to narrow the mutation range or adopt the mutation range recommended by the module for screening. In the calculation process, the calculation module preprocesses the candidate mutation amino acid sequences one by one and calculates and records the affinity of antibody antigens one by one based on the deep learning model. After the calculation is completed, the module scores and orders all candidate antibody sequences, and the N sequences with the highest affinity (the modification direction is enhanced) or the lowest affinity (the modification direction is weakened) may be the final modification sequence, wherein N is the number of user-defined sequences, and the default output sequence number is 200.

S3, the visual analysis display module 444 accepts all antibody/protein candidate modification sequences generated by the design module. The visual analysis display module 444 provides the information of the complete antibody sequence, and at the same time, provides the mutation sites comparison between template sequences and candidate modified sequences, and statistical chart of mutation sites, such as mutation sites contained in CDRH1, H2 and H3 regions of the antibody, respectively. In addition, the display of mutation sites thermal map is provided, including the original amino acid type of each mutation site and the amino acid type after mutation. In addition, the species of the mutated amino acids are also displayed. Classification and group mainly consider the physical and chemical properties of amino acids, and is divided into five groups: polar, nonpolar, aromatic, positively charged and negatively charged.

In addition, relying on the algorithm design and efficient computing resource allocation method, the present invention can search the mutation space of antibody/protein 1010 in a single time, breaking through the imagination barrier and calculation barrier in the traditional design, and allowing users to find the optimal solution for specific antigen in the super-large mutation space, so as to improve the hit rate and strength of affinity maturity.

More importantly, the virtual affinity module of the present invention adopts a fully automatic calculation process, and the calculation process and calculation method are not limited to one target or a certain kind of target. In addition, the virtual screening speed of the module is increased (the screening of one billion-level mutation spaces takes hours as a unit), and multiple affinity modification conditions of multiple targets can be simultaneously screened, which has important auxiliary significance for new drug and multi-target drug research and development.

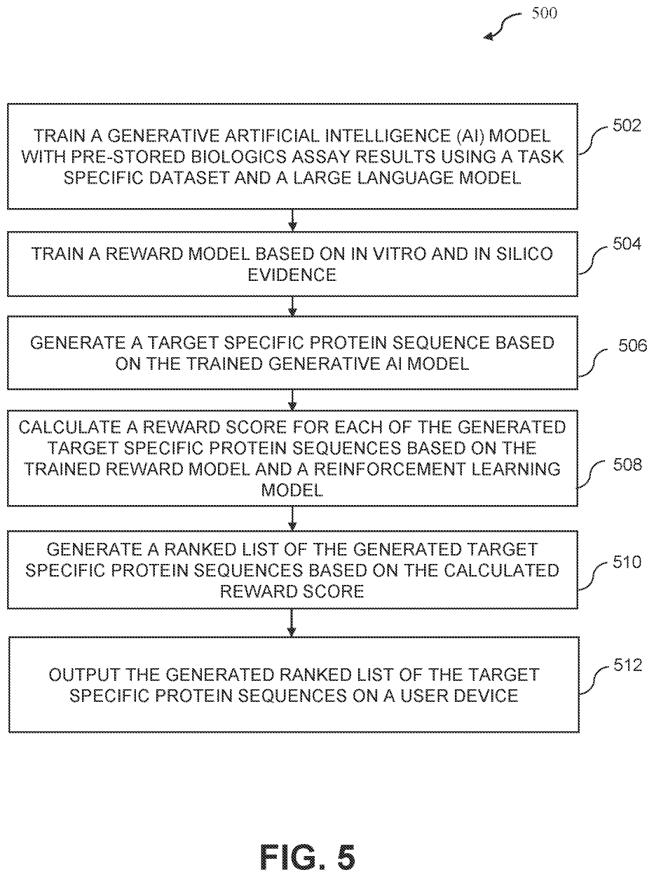

FIG. 5 illustrates a flow chart depicting a method 500 of designing protein sequences, in accordance with the embodiment of the present disclosure.

At block 502, the method 500 may include training, by one or more hardware processors 110, a generative artificial intelligence (AI) model with pre-stored biologics assay results using a task specific dataset and a large language model. The task specific dataset comprises a plurality of protein sequences.

At block 504, the method 500 may include training, by the one or more hardware processors 110, a reward model based on in vitro and in silico evidence.

At block 506, the method 500 includes generating, by the one or more hardware processors, a target specific protein sequences based on the trained generative AI model.

At block 508, the method 500 includes calculating, by the one or more hardware processors 110, a reward score for each of the generated target specific protein sequences based on the trained reward model and a reinforcement learning model.

At block 510, the method 500 includes generating, by the one or more hardware processors 110, a ranked list of the generated target specific protein sequences based on the calculated reward score.

At block 512, the method 500 includes outputting, by the one or more hardware processors 110, the generated ranked list of the target specific protein sequences on a user device.

The method 500 may be implemented in any suitable hardware, software, firmware, or combination thereof. The order in which the method 500 is described is not intended to be construed as a limitation, and any number of the described method blocks may be combined or otherwise performed in any order to implement the method 500 or an alternate method. Additionally, individual blocks may be deleted from the method 500 without departing from the spirit and scope of the present disclosure described herein. Furthermore, the method 500 may be implemented in any suitable hardware, software, firmware, or a combination thereof, that exists in the related art or that is later developed. The method 500 describes, without limitation, the implementation of the system 102. A person of skill in the art will understand that method 500 may be modified appropriately for implementation in various manners without departing from the scope and spirit of the disclosure.

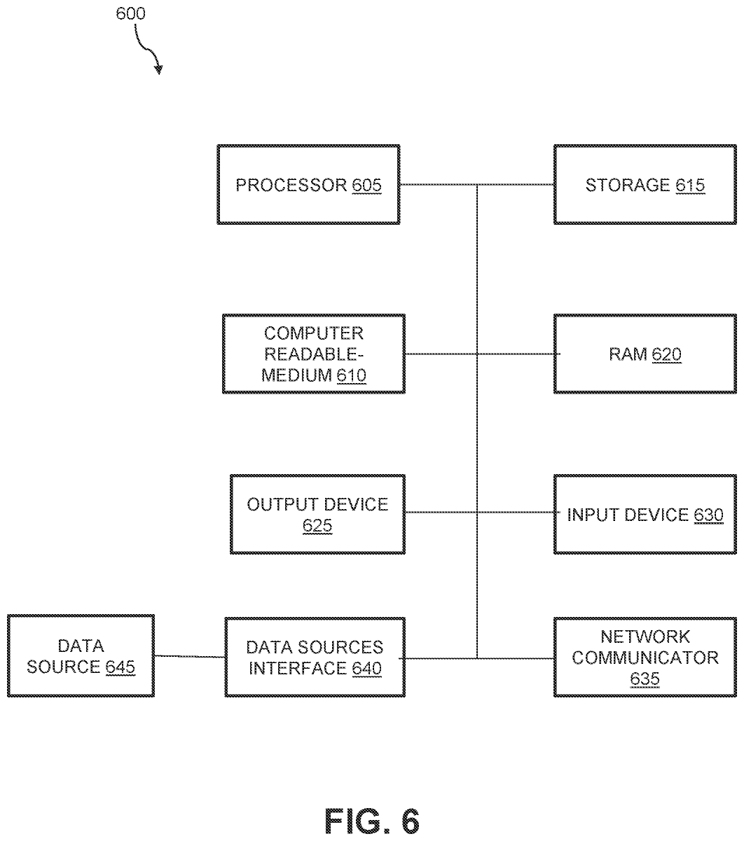

FIG. 6 illustrates an exemplary block diagram representation of a hardware platform 500 for implementation of the disclosed system 102, according to an example embodiment of the present disclosure. For the sake of brevity, the construction, and operational features of the system 102 which are explained in detail above are not explained in detail herein. Particularly, computing machines such as but not limited to internal/external server clusters, quantum computers, desktops, laptops, smartphones, tablets, and wearables may be used to execute the system 102 or may include the structure of the hardware platform 600. As illustrated, the hardware platform 600 may include additional components not shown, and some of the components described may be removed and/or modified. For example, a computer system with multiple GPUs may be located on external-cloud platforms including Amazon Web Services, internal corporate cloud computing clusters, or organizational computing resources.

The hardware platform 600 may be a computer system such as the system 102 that may be used with the embodiments described herein. The computer system may represent a computational platform that includes components that may be in a server or another computer system. The computer system may be executed by the processor 605 (e.g., single, or multiple processors) or other hardware processing circuits, the methods, functions, and other processes described herein. These methods, functions, and other processes may be embodied as machine-readable instructions stored on a computer-readable medium, which may be non-transitory, such as hardware storage devices (e.g., RAM (random access memory), ROM (read-only memory), EPROM (erasable, programmable ROM), EEPROM (electrically erasable, programmable ROM), hard drives, and flash memory). The computer system may include the processor 605 that executes software instructions or code stored on a non-transitory computer-readable storage medium 610 to perform methods of the present disclosure. The software code includes, for example, instructions to gather data and analyze the data. For example, the plurality of modules 114 includes a generative artificial intelligence (AI) module 206, a reward model generation module 208, a reinforcement learning module 210, and an output module 212.

The instructions on the computer-readable storage medium 610 are read and stored the instructions in storage 615 or random-access memory (RAM). The storage 615 may provide a space for keeping static data where at least some instructions could be stored for later execution. The stored instructions may be further compiled to generate other representations of the instructions and dynamically stored in the RAM such as RAM 620. The processor 605 may read instructions from the RAM 520 and perform actions as instructed.

The computer system may further include the output device 625 to provide at least some of the results of the execution as output including, but not limited to, visual information to users, such as external agents. The output device 625 may include a display on computing devices and virtual reality glasses. For example, the display may be a mobile phone screen or a laptop screen. GUIs and/or text may be presented as an output on the display screen. The computer system may further include an input device 630 to provide a user or another device with mechanisms for entering data and/or otherwise interacting with the computer system. The input device 630 may include, for example, a keyboard, a keypad, a mouse, or a touchscreen. Each of these output devices 625 and input device 630 may be joined by one or more additional peripherals. For example, the output device 625 may be used to display the results such as bot responses by the executable chatbot.

A network communicator 635 may be provided to connect the computer system to a network and in turn to other devices connected to the network including other clients, servers, data stores, and interfaces, for example. A network communicator 635 may include, for example, a network adapter such as a LAN adapter or a wireless adapter. The computer system may include a data sources interface 640 to access the data source 645. The data source 645 may be an information resource. As an example, a database of exceptions and rules may be provided as the data source 645. Moreover, knowledge repositories and curated data may be other examples of the data source 645.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article.

Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limited, of the scope of the invention, which is outlined in the following claims.

1. A computer-implemented system for collaborative smart evidence gathering and investigation for incident response, attack surface management, and forensics in a computing environment, the computer-implemented system comprising:
one or more processors;
a memory coupled to the one or more processors, wherein the memory comprises a plurality of modules in form of programmable instructions executable by the one or more processors, and wherein the plurality of modules comprises:
a data obtaining module configured to obtain evidence data corresponding to one or more events from multiple data sources, multimodal and multi-context entries, and initiation points, wherein the evidence data comprises one or more parameters and contextual information;
a data processing module configured to process the obtained evidence data into one or more investigation categories based on the one or more parameters and the contextual information using an artificial intelligence (AI) root cause analysis, graph augmented retrieval, semantic classifier, meaning extraction, and causal discovery model;
an analysis performing module configured to perform similarity analysis for the processed evidence data based on the one or more parameters and the contextual information;
an evidence evaluating module configured to evaluate an evidence quality, an evidence sufficiency, and an evidence completeness of the processed evidence data based on the performed similarity analysis, intrinsic factors, and extrinsic inputs;
an action determining module configured to determine one or more actions to be performed on the processed evidence data based on the evaluated evidence quality, the evidence sufficiency, and the evidence completeness; and
an action performing module configured to perform the determined one or more actions on the processed evidence data to resolve the one or more events.

2. The computer-implemented system of claim 1, wherein for determining one or more actions to be performed on the processed evidence data based on the evaluated evidence quality, the evidence sufficiency, and the evidence completeness, the plurality of modules further comprises:
a data storing module configured to store the processed evidence data into at least one of a bookmark library, collectible library, and a historical evidence library; and
a data assigning module configured to assign the processed evidence data to at least one of an existing case and a new case based on manifestation of the evidence data.

3. The computer-implemented system of claim 1, wherein for determining one or more actions to be performed on the processed evidence data based on the evaluated evidence quality, the evidence sufficiency, and the evidence completeness, the plurality of modules further comprises:
an evidence type determining module configured to determine a type of required additional evidence to support investigation based on the evaluated evidence quality, the evidence sufficiency, and the evidence completeness.

4. The computer-implemented system of claim 1, wherein for determining one or more actions to be performed on the processed evidence data based on the evaluated evidence quality, the evidence sufficiency, and the evidence completeness, the plurality of modules further comprises:
a replay determining module configured to determine the evidence to replay;
a data retrieving module configured to retrieve dataset and visual representation information associated with the determined evidence;
a preview generating module configured to generate an embedded preview of the determined evidence for replaying the determined evidence; and
a state assessing module configured to assess state of the determined evidence before, during and after a context when the determined evidence originally appeared.

5. The computer-implemented system of claim 1, wherein for determining one or more actions to be performed on the processed evidence data based on the evaluated evidence quality, the evidence sufficiency, and the evidence completeness, the plurality of modules further comprises:
an evidence inferring module configured to infer additional evidence required to be gathered as a support to the obtained evidence data based on the evaluated evidence quality, the evidence sufficiency, and the evidence completeness;
an evidence obtaining module configured to obtain the inferred additional evidence by defining search parameters corresponding to the inferred additional evidence;
an evidence simulating module configured to simulate the obtained additional evidence to determine the evidence quality, the evidence sufficiency, and the evidence completeness of the additional evidence;
a pathway determining module configured to determine exploitation pathway for the additional evidence using generative and adversarial AI models;
a missing data determining module configured to determine missing data in the additional evidence based on the determined exploitation pathway, determined evidence quality, the evidence sufficiency, and the evidence completeness of the additional evidence; and
an evidence refining data configured to refine the additional evidence to recreate the determined missing data.

6. The computer-implemented system of claim 1, wherein for determining one or more actions to be performed on the processed evidence data based on the evaluated evidence quality, the evidence sufficiency and the evidence completeness, the plurality of modules further comprises:
a root cause determining module configured to determine possible root causes for the additional evidence;
a weight assigning module configured to assign likelihood weights to each of the determined possible root causes; and
a state determining module configured to determine state of investigation of the one or more events based on the assigned likelihood weights.

7. The computer-implemented system of claim 1, wherein for determining one or more actions to be performed on the processed evidence data based on the evaluated evidence quality, the evidence sufficiency and the evidence completeness, the plurality of modules further comprises:
a query triggering module configured to trigger additional questions and queries associated with the obtained evidence data for improving evidence quality.

8. The computer-implemented system of claim 1, wherein the plurality of modules further comprises:
a participation enabling module configured to enable participation of a computer generated smart expert system as a member of evidence gathering and investigation team;
a decision generating module configured to generate collective decisions on the one or more events based on decisions collected from the computer generated smart expert system;
a workflow communication enabling module configured to enable threaded conversation and workflow centric resolution between the computer generated smart expert system to resolve the one or more events; and
a first class investigator configured to manage investigation process state over time to time for the one or more events.

9. The computer-implemented system of claim 1, wherein the one or more events correspond to at least one of security and operational incidents, proactive attack surface management, and post facto forensics.

10. The computer-implemented system of claim 1, wherein the plurality of modules further comprises:
an evidence managing module configured to perform at least one of evidence ordering, sorting, stitching, and weighting for determination of attack paths, attack vectors, indicators of compromise, vulnerability impact, exploitable entry points, and security centric blast radius and impact zone calculations.

11. A computer-implemented method for collaborative smart evidence gathering and investigation for incident response, attack surface management, and forensics in a computing environment, the computer-implemented method comprising:
obtaining, by one or more processors, evidence data corresponding to one or more events from multiple data sources, multimodal and multi-context entries, and initiation points, wherein the evidence data comprises one or more parameters and contextual information, and wherein the one or more events correspond to at least one of security and operational incidents, proactive attack surface management, and post facto forensics;
processing, by the one or more processors, the obtained evidence data into one or more investigation categories based on the one or more parameters and contextual information using an artificial intelligence (AI) root cause analysis, graph augmented retrieval, semantic classifier, meaning extraction, and causal discovery model;
performing, by the one or more processors, similarity analysis for the processed evidence data based on the one or more parameters and the contextual information;
evaluating, by the one or more processors, an evidence quality, an evidence sufficiency, and an evidence completeness of the processed evidence data based on the performed similarity analysis, intrinsic factors, and extrinsic inputs;
determining, by the one or more processors, one or more actions to be performed on the processed evidence data based on the evaluated evidence quality, the evidence sufficiency, and the evidence completeness; and
performing, by the one or more processors, the determined one or more actions on the processed evidence data to resolve the one or more events.

12. The computer-implemented method of claim 11, wherein determining one or more actions to be performed on the processed evidence data based on the evaluated evidence quality, the evidence sufficiency, and the evidence completeness, further comprises:
storing, by the one or more processors, the processed evidence data into at least one of a bookmark library, collectible library, and a historical evidence library; and
assigning, by the one or more processors, the processed evidence data to at least one of an existing case and a new case based on manifestation of the evidence data.

13. The computer-implemented method of claim 11, wherein determining one or more actions to be performed on the processed evidence data based on the evaluated evidence quality, the evidence sufficiency, and the evidence completeness, further comprises:
determining, by the one or more processors, type of required additional evidence to support investigation based on the evaluated evidence quality, the evidence sufficiency, and the evidence completeness.

14. The computer-implemented method of claim 11, wherein determining one or more actions to be performed on the processed evidence data based on the evaluated evidence quality, the evidence sufficiency, and the evidence completeness, further comprises:
determining, by the one or more processors, the evidence to replay;
retrieving, by the one or more processors, dataset and visual representation information associated with the determined evidence;
generating, by the one or more processors, an embedded preview of the determined evidence for replaying the determined evidence; and
assessing, by the one or more processors, state of the determined evidence before, during and after a context when the determined evidence originally appeared.

15. The computer-implemented method of claim 11, wherein determining one or more actions to be performed on the processed evidence data based on the evaluated evidence quality, the evidence sufficiency, and the evidence completeness, further comprises:
inferring, by the one or more processors, additional evidence required to be gathered as a support to the obtained evidence data based on the evaluated evidence quality, the evidence sufficiency, and the evidence completeness;
defining, by the one or more processors, search parameters corresponding to the inferred additional evidence;
simulating, by the one or more processors, the obtained additional evidence to determine the evidence quality, the evidence sufficiency, and the evidence completeness of the additional evidence;
determining, by the one or more processors, exploitation pathway for the additional evidence using generative and adversarial AI models;
determining, by the one or more processors, missing data in the additional evidence based on the determined exploitation pathway, determined evidence quality, the evidence sufficiency, and the evidence completeness of the additional evidence; and
refining, by the one or more processors, the additional evidence to recreate the determined missing data.

16. The computer-implemented method of claim 11, wherein determining one or more actions to be performed on the processed evidence data based on the evaluated evidence quality, the evidence sufficiency, and the evidence completeness, further comprises:
determining, by the one or more processors, possible root causes for the additional evidence;
assigning, by the one or more processors, likelihood weights to each of the determined possible root causes; and
determining, by the one or more processors, state of investigation of the one or more events based on the assigned likelihood weights.

17. The computer-implemented method of claim 11, wherein determining one or more actions to be performed on the processed evidence data based on the evaluated evidence quality, the evidence sufficiency, and the evidence completeness, further comprises:
triggering, by the one or more processors, additional questions and queries associated with the obtained evidence data for improving evidence quality.

18. The computer-implemented method of claim 11, further comprising:
enabling by the one or more processors, participation of a computer generated smart expert system as a member of evidence gathering and investigation team;
generating, by the one or more processors, collective decisions on the one or more events based on decisions collected from the computer generated smart expert system;
enabling, by the one or more processors, threaded conversation and workflow centric resolution between the computer generated smart expert system to resolve the one or more events; and
managing, by the one or more processors, investigation process state over time to time for the one or more events.

19. The computer-implemented method of claim 11, further comprising:
performing, by the one or more processors, at least one of evidence ordering, sorting, stitching, and weighting for determination of attack paths, attack vectors, indicators of compromise, vulnerability impact, exploitable entry points, and security centric blast radius and impact zone calculations.

20. A non-transitory computer-readable storage medium having instructions stored therein that, when executed by one or more processors, cause the one or more processors to:
obtain evidence data corresponding to one or more events from multiple data sources, multimodal and multi-context entries, and initiation points, wherein the evidence data comprises one or more parameters and contextual information;
process the obtained evidence data into one or more investigation categories based on the one or more parameters and contextual information using an artificial intelligence (AI) root cause analysis, graph augmented retrieval, semantic classifier, meaning extraction, and causal discovery model;
perform similarity analysis for the processed evidence data based on the one or more parameters and the contextual information;
evaluate an evidence quality, an evidence sufficiency, and an evidence completeness of the processed evidence data based on the performed similarity analysis, intrinsic factors, and extrinsic inputs;
determine one or more actions to be performed on the processed evidence data based on the evaluated evidence quality, the evidence sufficiency, and the evidence completeness; and
perform the determined one or more actions on the processed evidence data to resolve the one or more events.
